(19) United States
(12) Patent Application Publication
(10) Pub. No.: US 2021/0155931 A1
(43) Pub. Date: May 27, 2021

(54) TREATMENT OF RAPIDLY EVOLVING BIOLOGICAL ENTITIES

(71) Applicant: Augmanity Nano LTD, Rehovot (IL)

(72) Inventors: Ido Bachelet, Tel Aviv- Yafo (IL); Erez Lavi, Rehovot (IL); Sapir Sasson, Holon (IL); Liron A. Bassali, Givat Shmuel (IL); Elinor Debby, Herzliya (IL); Anastasia Shapiro, Rehovot (IL); Itai Rusinek, Rehovot (IL); Gil Harai, Rehovot (IL); Danielle Karo-Atar, Ramla (IL); Noam Mamet, Dolev (IL); Yaniv Amir, Tel Aviv-Yafo (IL); Almogit Abu-Horowitz, Herzliya (IL)

(21) Appl. No.: 16/612,161

(22) PCT Filed: May 7, 2018
(86) PCT No.: PCT/IB18/00613

§ 371 Date: Nov. 8, 2019

Related U.S. Application Data
(60) Provisional application No. 62/503,074, filed on May 8, 2017.

(51) Int. Cl. C12N 15/115; C12Q 1/6883

(52) U.S. Cl. CPC C12N 15/115; G01N 2800/52; C12N 2310/16; C12Q 1/6883

(57) ABSTRACT

The present disclosure describes methods and compositions for the treatment of rapidly evolving biological entities (e.g., cancer cells, bacteria, virus, etc.) using therapeutic nucleic acids.

### RELATED APPLICATIONS

This application is a § 371 national-stage application based on PCT Application number PCT/IB18/00613, filed May 7, 2018, which claims the benefit of priority to U.S. Provisional Patent Application Ser. No. 62/503,074, filed May 8, 2017, each of which is hereby incorporated by reference in its entirety.

### SEQUENCE LISTING

This application contains a Sequence Listing which has been submitted electronically in ASCII format and is hereby incorporated by reference in its entirety. Said ASCII copy, created on Sep. 4, 2018, is named ANB-00225 SL.txt and is 1,271 bytes in size.

### BACKGROUND

Cancer, bacteria, and viruses are all major threats to human health, and impose a severe burden on the global economy. Although very different, cancer, bacteria, and viruses share one critical factor—their ability to evolve and acquire drug resistance. It is clear that rapidly-evolving targets such as theses cannot be effectively countered with a single “magic bullet”. However, the discovery of new drugs to treat these diseases using conventional methodologies can take years. Thus, new more rapid and cost-effective methods for the development of new treatments are needed to produce new therapeutics against these targets.

Aptamers are short, single-stranded nucleic acid oligomers that can bind to a specific target molecule and/or exert effects on it. Aptamers are typically selected from a large random pool of oligonucleotides in an iterative process. More recently, aptamers have been successfully selected in cells, in-vivo and in-vitro.

The selection of aptamers, their structure-function relationship, and their mechanisms of action are all poorly-understood. Although more than 100 aptamer structures have been solved and reported, almost no recurring structural motifs have been identified.

A variety of different aptamer selection processes have been described for identifying aptamers capable of binding to a particular target. However, the ability to rapidly and conveniently identify aptamers able to mediate a desirable functional effect on a target of interest would have a profound impact on aptamer therapeutics and on the treatment of rapidly evolving diseases.

### SUMMARY

The present disclosure relates to compositions and methods for the treatment diseases and disorders caused by rapidly evolving biological entities (e.g., cancer, bacterial infections, viral infections, fungal infections, etc.). The methods disclosed herein allow for the rapid development of novel therapeutics (e.g., target-specific aptamers) for treating diseases or conditions associated with a rapidly evolving target (e.g., cancer, bacterial infection, viral infection, fungal infections, etc. etc.).

In certain aspects, provided herein are methods for treating a subject (e.g., a human subject) for a disease associated with a rapidly evolving biological entity (e.g., cancer, bacterial infection, viral infection, fungal infection, etc.). In some embodiments, the methods comprise (a) administering to the subject a therapeutic nucleic acid (e.g., an aptamer or an interfering RNA) that targets the rapidly evolving biological entity (e.g., a cancer cell, a bacterium, a virus); (b) determining whether the subject exhibits a therapeutic response; and (c) if the subject fails to demonstrate a therapeutic response, then obtaining a sample from the subject comprising the rapidly evolving biological entity, performing a screening assay to identify a new therapeutic nucleic acid that targets the rapidly evolving biological entity, and administering to the subject the new therapeutic nucleic acid. In some embodiments, step (c) of the methods also includes continuing to administer the therapeutic nucleic acid if the subject shows a therapeutic response. In some embodiments, the therapeutic nucleic acid is a nucleic acid aptamer.

In some embodiments, steps (b)-(c) of the methods are repeated (e.g., repeated at least 2, 3, 4, 5, 6, 7, 8, 9 or 10 times). In some embodiments, steps (b)-(c) are repeated until the disease associated with the rapidly evolving biological entity is treated and/or the rapidly evolving biological entity is eliminated in the subject.

In some embodiments, the methods further comprise (1) obtaining a sample from the subject comprising the rapidly evolving biological entity; (2) performing a screening assay to identify a therapeutic nucleic acid that targets the rapidly evolving biological entity prior to step (a). In some embodiments, the methods further comprise performing an analysis of the rapidly evolving biological entity prior to step (a).

In certain aspects, disclosed herein are methods for treating a subject (e.g., a human subject) for a disease associated with a rapidly evolving biological entity (e.g., cancer, bacterial infection, virus infection, fungal infection, etc.) wherein the methods comprise (a) administering to the subject a therapeutic nucleic acid that targets the rapidly evolving biological entity (e.g., an aptamer or an interfering RNA); (b) after a period of time, obtaining a sample from the subject comprising the rapidly evolving biological entity; (c) performing a screening assay to identify a new therapeutic nucleic acid that targets the rapidly evolving biological entity; and (d) administering to the subject the new therapeutic nucleic acid. In some embodiments, the therapeutic nucleic acid is a nucleic acid aptamer.

In some embodiments, the period of time in step (b) is equal to or shorter than the period of time required for the rapidly evolving biological entity to acquire resistance to the first therapeutic nucleic acid. In some embodiments, the period of time in step (b) is equal to or shorter than the period of time required for the rapidly evolving biological entity to complete a replication cycle. In some embodiments, the period of time is at least 20 minutes, 30 minutes, 40 minutes, 50 minutes, 1 hour, 2 hours, 3 hours, 4 hours, 5 hours, 6 hours, 12 hours, 1 day, 2 days, 3 days, 4 days, 5 days, 6 days, 7 days. 8 days, 9 days. 10 days, 11 days, 12 days, 13 days, 2 weeks, 3 weeks, 4 weeks, 1 month, 2 months, 3 months, 4 months, 5 months or 6 months. In certain embodiments, the period of time is no more than 6 hours, 12 hours, 1 day, 2 days, 3 days, 4 days, 5 days, 6 days, 7 days. 8 days, 9 days. 10 days, 11 days, 12 days, 13 days, 2 weeks, 3 weeks, 4 weeks, 1 month, 2 months, 3 months, 4 months, 5 months or 6 months. In certain embodiments, the period of time is about 6 hours, 12 hours, 1 day, 2 days, 3 days, 4 days, 5 days, 6 days, 7 days. 8 days, 9 days. 10 days, 11 days, 12 days, 13 days, 2 weeks, 3 weeks, 4 weeks, 1 month, 2 months, 3 months, 4 months, 5 months or 6 months.

In some embodiments, steps (b)-(d) of the methods are repeated (e.g., repeated at least 2, 3, 4, 5, 6, 7, 8, 9 or 10 times). In some embodiments, steps (b)-(d) are repeated until the disease associated with the rapidly evolving biological entity is treated and/or the rapidly evolving biological entity is eliminated in the subject.

In some embodiments, the methods further comprise (1) obtaining a sample from the subject comprising the rapidly evolving biological entity; (2) performing a screening assay to identify a therapeutic nucleic acid that targets the rapidly evolving biological entity prior to step (a). In some embodiments, the methods further comprise performing an analysis of the rapidly evolving biological entity prior to step (a).

In certain embodiments, the methods further comprise identifying one or more aptamers that specifically bind to the rapidly evolving biological entity. In some embodiments, the methods comprise (i) contacting a plurality of aptamer clusters immobilized on a surface (e.g., a flow cell surface) with the rapidly evolving biological entity; and (ii) identifying immobilized aptamer clusters that bind to the rapidly evolving biological entity. In certain embodiments, the methods further comprise performing a wash step after step (i) to remove unbound rapidly evolving biological entity from surface (e.g., a flow cell surface). In some embodiments, the rapidly evolving biological entity is detectably labeled (e.g., fluorescently labeled).

In some embodiments, the methods comprise identifying one or more aptamers that modulate a property of the rapidly evolving biological entity. In some embodiments, the methods comprise (i) contacting a plurality of aptamer clusters immobilized on a surface with the rapidly evolving biological entity; and (ii) identifying the immobilized aptamer clusters that modulate the property of the rapidly evolving biological entity (e.g., cell viability, cell proliferation, gene expression, cell morphology, etc.). In some embodiments, the methods further comprise performing a wash step after step (i) to remove unbound rapidly evolving biological entity from surface (e.g., a flow cell surface). In some embodiments, the rapidly evolving biological entity comprises a detectable label (e.g., a fluorescent dye, such as a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, or a redox potential dye). In some embodiments, a change in the property of the rapidly evolving biological entity causes a change in the properties of the detectable label which are detected in order to identify the immobilized aptamer clusters that modulate the property of the rapidly evolving biological entity.

In certain embodiments, the methods further comprise the generation of the immobilized aptamer clusters. In some embodiments, the immobilized aptamer clusters are generated by: (a) immobilizing a plurality of aptamers (e.g., from an aptamer library) on the surface; and (b) amplifying the plurality of immobilized aptamers locally on the flow cell surface (e.g., via bridge PCR amplification or rolling circle amplification) to form the plurality of immobilized aptamer clusters. In some embodiments, the methods further comprise removing the complementary strands from the immobilized aptamer clusters to provide single stranded immobilized aptamer clusters. In certain embodiments, the immobilized aptamer clusters are sequenced following step (b) (e.g., using Illumina sequencing or Polonator sequencing). In some embodiments, the immobilized aptamer clusters are generated by printing aptamer clusters (e.g., from an aptamer library) directly on the surface. In some embodiments, the methods comprise the generation of the aptamer library (e.g., through chemical nucleic acid synthesis).

### BRIEF DESCRIPTION OF FIGURES

FIG. 1 has two panels. Panel A is a schematic diagram of the process for treating a disease associated with a rapidly evolving biological entity, which includes continuous sampling, target analysis, agent selection, treatment and effect work flow according to certain embodiments described herein. Panel B is a schematic diagram of the process for treating a disease associated with a rapidly evolving biological entity, which includes sampling, analysis, and agent selection work flow according to certain embodiments described herein.

FIG. 2 is a schematic representation of the process for treating a disease associated with a rapidly evolving biological entity which includes target growth/onset of disease or condition, selection of agent, acquisition of target resistance, selection of a second agent for treatment, acquisition of a second resistance, and selection of a third agent according to certain embodiments described herein.

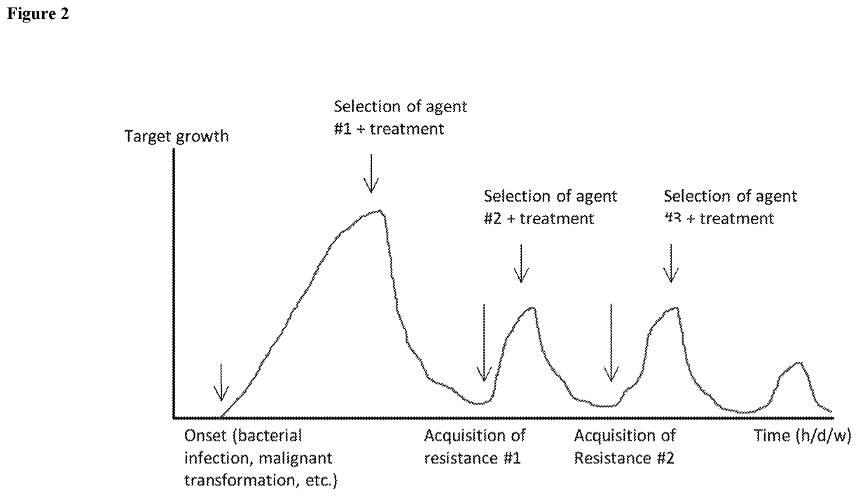

FIG. 3 is a schematic diagram of aptamer library synthesis, sequencing and target identification work flow according to certain embodiments described herein.

FIG. 4 is a bar graph showing the binding of target cells (Hana cells) to a library of aptamers (Lib), short or long aptamers of random sequence, aptamer outputs of SELEX selection process for the specific target cells cycles 6 and 7 (Cyc6 and Cyc7 respectively), specific aptamer sequences from SELEX selection process (Apt1 and Apt2), and an empty lane (empty) on an Illumina GAIIx flow-cell. Cells were run down flow cell lanes, and bound cells counted (bound vs. unbound, expressed as fraction, 1=100% of cells).

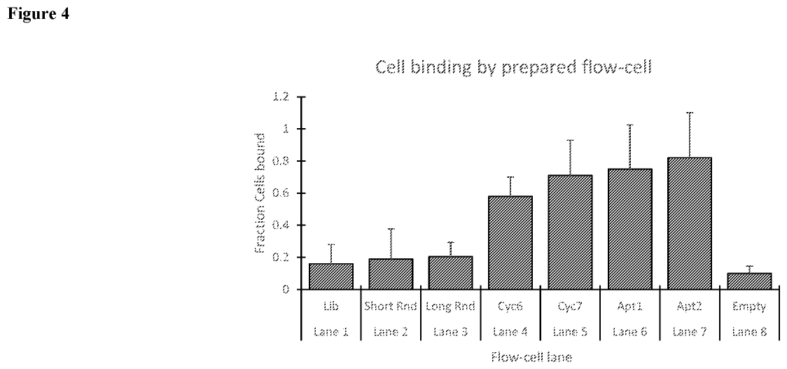

FIG. 5 is an image of a cell bound to aptamers on a flow cell. The image shows the movement of the cell relative to the surface over time. The image shows that the cell is retained by the immobilized aptamer cluster, rather than attached to the surface itself, and is thus free to move but confined to that location. Imaging was performed on an Illumina GAIIx.

FIG. 6 is a schematic representation of certain aptamer structures according to certain exemplary embodiments provided herein.

### DETAILED DESCRIPTION

### General

The present disclosure relates to any nucleic acid-based therapy of rapidly-evolving targets (e.g., cancer, bacterial infections, viral infections, fungal infections, etc.) that relies on a repetitive process for developing a new therapeutic agent or agents against new mutated versions of a rapidly-evolving target. In some embodiments, the selection process is defined by f_{Ther}≥f_{TE}, where f_{Ther} is frequency of therapeutic selection and f_{TE} is frequency of target evolution or more precisely frequency of acquiring resistance by the target. The agents of the present disclosure (e.g., therapeutic nucleic acids disclosed herein) can be selected for any function (e.g., binding, cytotoxicity, growth inhibition, binding to specific membrane or capsule molecules, anti-quorum sensing, etc.) against the target following every phenotypic change it undergoes, or at steady time intervals.

Provided herein are methods and composition related for the treatment of rapidly evolving biological entity (e.g., cancer, bacterial infection, virus infection, etc.) using aptamers that bind to and/or mediate a functional effect on a target (e.g., a target cell or a target molecule).

In some embodiments, the present disclosure relates to methods for treating a subject for a disease associated with a rapidly evolving biological entity (e.g., cancer, bacterial infection, virus infection, etc.). In some embodiments, the methods comprise administering to the subject a first therapeutic nucleic acid that targets the rapidly evolving biological entity and determining whether the subject exhibits a therapeutic response to the first therapeutic nucleic acid, In some embodiment, if the subject fails to demonstrate a therapeutic response to the first therapeutic nucleic acid a sample is obtained from the subject comprising the rapidly evolving biological entity, a screening assay is performed to identify a second therapeutic nucleic acid that targets the rapidly evolving biological entity, and the subject is administered the second therapeutic nucleic acid. In some embodiments, the methods further comprise continuing to administer the first therapeutic nucleic acid if the subject if the subject shows a therapeutic response to the first therapeutic nucleic acid.

In some aspects, the methods are repeated until the disease associated with the rapidly evolving biological entity is treated. In some embodiments, the methods further comprise performing an analysis of the rapidly evolving biological entity prior to administering to the subject a first therapeutic nucleic acid that targets the rapidly evolving biological entity. In some embodiments, the therapeutic nucleic acid is an interfering RNA or a nucleic acid aptamer. In one embodiment, the therapeutic nucleic acid is a nucleic acid aptamer.

In other aspects, the methods comprise administering to the subject a first therapeutic nucleic acid that targets the rapidly evolving biological entity, obtaining a sample from the subject after a period of time comprising the rapidly evolving biological entity. In some embodiments, a screening assay is performed to identify a second therapeutic nucleic acid that targets the rapidly evolving biological entity and the second therapeutic nucleic acid is administered to the subject. In some embodiments, the therapeutic nucleic acid is an aptamer.

In some embodiments, the present disclosure relates to aptamers (DNA, RNA, or any natural or synthetic analog of these), and methods for rapidly selecting target-specific aptamers for the treatment of rapidly-evolving targets.

In certain embodiments, the sequence of each immobilized aptamer cluster is known and/or determined, for example, by sequencing the aptamer clusters or by printing aptamers of known sequences onto predetermined positions of the surface. Thus, by determining the position on the surface at which the rapidly evolving biological entity binds to, interacts with and/or is modulated by an aptamer cluster, the relevant effect can be associated with the aptamer sequence at that position.

For example, in some embodiments, aptamers that bind to the rapidly evolving biological entity are identified by running a composition comprising the rapidly evolving biological entity that comprises a detectable label (e.g., a fluorescent label) across a surface to which aptamer clusters of known sequences are immobilized at known positions. The positions on the surface at which the rapidly evolving biological entity is retained are determined (e.g., using fluorescent microscopy), indicating that the aptamers immobilized at those positions bind to the target.

In certain embodiments, aptamers that functionally modulate the rapidly evolving biological entity are identified by running a composition comprising the rapidly evolving biological entity that comprises a detectable label indicative of the function being modulated (e.g., a fluorescent dye, such as a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, or a redox potential dye) across a surface to which aptamer clusters of known sequences are immobilized at known positions. The positions on the surface at which the detectable label indicates that the rapidly evolving biological entity is modulated are determined (e.g., using fluorescent microscopy), indicating that the aptamers immobilized at those positions are able to modulate the rapidly evolving biological entity.

In certain aspects, also provided herein are methods and compositions related to the creation of immobilized of aptamer clusters on a surface. In some embodiments, aptamers (e.g., from an aptamer library disclosed herein) are immobilized onto a surface, such as a flow cell surface. In some embodiments, a localized amplification process, such as bridge amplification or rolling circle amplification, is then performed to generate aptamer clusters. The aptamer clusters can then be sequenced (e.g., by Illumina sequencing or Polonator sequencing) in order to associate the sequence of each aptamer cluster with a position on the surface. The complementary strands can be stripped in order to generate single-stranded aptamer clusters. The surface (e.g., flow cell) is then ready for use in an aptamer identification method provided herein.

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

As used herein, the term “aptamer” refers to a short (e.g., less than 200 bases), single stranded nucleic acid molecule (ssDNA and/or ssRNA) able to specifically bind to a protein or peptide target.

As used herein, the term “aptamer cluster” refers to a collection of locally immobilized aptamers (e.g., at least 10) of identical sequence.

The term “binding” or “interacting” refers to an association, which may be a stable association, between two molecules, e.g., between an aptamer and target, e.g., due to, for example, electrostatic, hydrophobic, ionic and/or hydrogen-bond interactions under physiological conditions.

As used herein, two nucleic acid sequences “complement” one another or are “complementary” to one another if they base pair one another at each position.

As used herein, two nucleic acid sequences “correspond” to one another if they are both complementary to the same nucleic acid sequence.

As used herein, the terms “interfering RNA molecule”, “inhibiting RNA molecule” and “RNAi molecule” are used interchangeably. Interfering RNA molecules include, but are not limited to, siRNA molecules, single-stranded siRNA molecules and shRNA molecules. Interfering RNA molecules generally act by forming a heteroduplex with the target molecule, which is selectively degraded or “knocked down,” hence inactivating the target RNA. Under some conditions, an interfering RNA molecule can also inactivate a target transcript by repressing transcript translation and/or inhibiting transcription of the transcript.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity, or process. In certain instances, such regulation may be contingent on the occurrence of a specific event, such as activation of a signal transduction pathway, and/or may be manifest only in particular cell types.

A “patient” or “subject” refers to either a human or a non-human animal.

As used herein, “specific binding” refers to the ability of an aptamer to bind to a predetermined target. Typically, an aptamer specifically binds to its target with an affinity corresponding to a K_{D} of about 10⁻⁷ M or less, about 10⁻⁸ M or less, or about 10⁻⁹ M or less and binds to the target with a K_{D} that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target (e.g., BSA, casein, or an unrelated cell, such as an HEK 293 cell or an E. coli cell).

As used herein, the Tm or melting temperature of two oligonucleotides is the temperature at which 50% of the oligonucleotide/targets are bound and 50% of the oligonucleotide target molecules are not bound. Tm values of two oligonucleotides are oligonucleotide concentration dependent and are affected by the concentration of monovalent, divalent cations in a reaction mixture. Tm can be determined empirically or calculated using the nearest neighbor formula, as described in Santa Lucia, J. PNAS (USA) 95:1460-1465 (1998), which is hereby incorporated by reference.

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

“Treating” a disease in a subject or “treating” a subject having a disease refers to subjecting the subject to a pharmaceutical treatment, e.g., the administration of a composition disclosed or contemplated herein, such that at least one symptom of the disease is decreased or prevented from worsening.

### Methods of Treatment

In certain aspects, provided herein are methods of treating diseases and disorders related to and/or caused by a rapidly evolving biological entity, such as a cancer cell, a bacterium, a virus and/or a fungus using therapeutic nucleic acids. In certain embodiments, the method leverages the methods provided herein for rapid identification of target-specific aptamers to adjust the therapeutic nucleic acid being administered to the subject to compensate for the evolution of the biological entity.

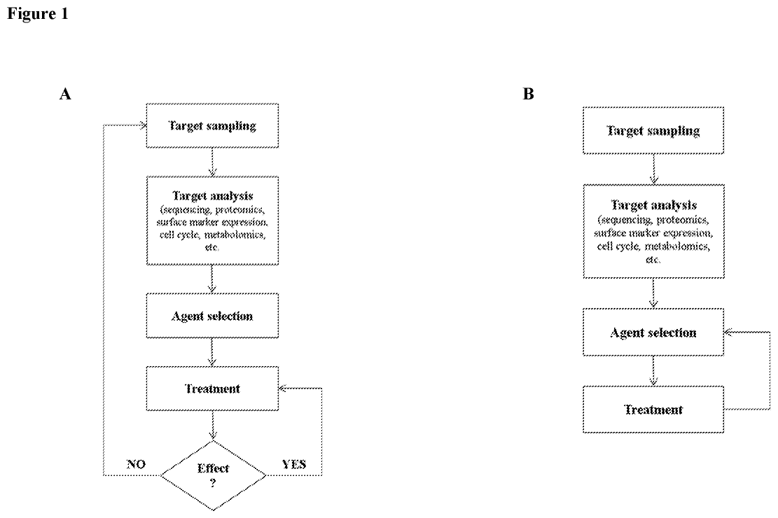

FIG. 1A provides a schematic diagram of an exemplary process for treating a disease associated with a rapidly evolving biological entity, which includes continuous sampling, target analysis, agent selection, treatment and effect work flow according to certain embodiments described herein. In certain embodiments, the methods and compositions provided herein relate to treating a subject for a disease associated with a rapidly evolving biological entity with an aptamer. The methods comprise (a) administering to the subject a first therapeutic nucleic acid that targets the rapidly evolving biological entity; (b) determining whether the subject exhibits a therapeutic response to the first therapeutic nucleic acid; and (c) if the subject fails to demonstrate a therapeutic response to the first therapeutic nucleic acid. In some embodiment, if the subject fails to demonstrate a therapeutic response to the first therapeutic nucleic acid the methods further comprise (i) obtaining a sample from the subject comprising the rapidly evolving biological entity; (ii) performing a screening assay to identify a second therapeutic nucleic acid that targets the rapidly evolving biological entity; and (iii) administering to the subject the second therapeutic nucleic acid. In some embodiments, if the subject shows a therapeutic response to the first therapeutic nucleic acid, administration of the first therapeutic nucleic acid is continued. In some embodiments, steps (b)-(c) are repeated until the disease associated with the rapidly evolving biological entity is treated. In some embodiments, steps (b)-(c) of the methods are repeated (e.g., repeated at least 2, 3, 4, 5, 6, 7, 8, 9 or 10 times). In some embodiments, steps (b)-(c) are repeated until the disease associated with the rapidly evolving biological entity is treated and/or the rapidly evolving biological entity is eliminated in the subject.

In some embodiments, the methods further comprise the step of performing a screening assay on a sample obtained from the subject to identify the first therapeutic nucleic acid prior to step (a). In some embodiments, the method further comprises obtaining the sample from the subject.

In some embodiments, the methods further comprise performing an analysis of the rapidly evolving biological entity prior to step (a). In one embodiment, the analysis of the rapidly evolving biological entity comprises a nucleic acid sequencing analysis, a proteomic analysis, a surface marker expression analysis, a cell cycle analysis, or a metabolomics analysis, or analysis by direct selection of the nucleic acid without a-priori knowledge of the entity's genotype and/or phenotype.

FIG. 1B provides a schematic diagram of an exemplary process for treating a disease associated with a rapidly evolving biological entity, which includes sampling, analysis, and agent selection work flow according to certain embodiments described herein. In certain embodiments, the methods provided herein relate to treating a subject for a disease associated with a rapidly evolving biological entity, the methods comprising: (a) administering to the subject a first therapeutic nucleic acid that targets the rapidly evolving biological entity; (b) after a period of time, obtaining a sample from the subject comprising the rapidly evolving biological entity; (c) performing a screening assay to identify a second therapeutic nucleic acid that targets the rapidly evolving biological entity; and (d) administering to the subject the second therapeutic nucleic acid.

In some embodiments, the period of time in step (b) is equal to or shorter than the period of time required for the rapidly evolving biological entity to acquire resistance to the first therapeutic nucleic acid. In some embodiments, the period of time in step (b) is equal to or shorter than the period of time required for the rapidly evolving biological entity to complete a replication cycle. In some embodiments, the period of time is at least 6 hours, 12 hours, 1 day, 2 days, 3 days, 4 days, 5 days, 6 days, 7 days. 8 days, 9 days. 10 days, 11 days, 12 days, 13 days, 2 weeks, 3 weeks, 4 weeks, 1 month, 2 months, 3 months, 4 months, 5 months or 6 months. In certain embodiments, the period of time is no more than 6 hours, 12 hours, 1 day, 2 days, 3 days, 4 days, 5 days, 6 days, 7 days. 8 days, 9 days. 10 days, 11 days, 12 days, 13 days, 2 weeks, 3 weeks, 4 weeks, 1 month, 2 months, 3 months, 4 months, 5 months or 6 months. In certain embodiments, the period of time is about 6 hours, 12 hours, 1 day, 2 days, 3 days, 4 days, 5 days, 6 days, 7 days. 8 days, 9 days. 10 days, 11 days, 12 days, 13 days, 2 weeks, 3 weeks, 4 weeks, 1 month, 2 months, 3 months, 4 months, 5 months or 6 months.

In some embodiments, steps (b)-(d) of the methods are repeated (e.g., repeated at least 2, 3, 4, 5, 6, 7, 8, 9 or 10 times). In some embodiments, steps (b)-(d) are repeated until the disease associated with the rapidly evolving biological entity is treated and/or the rapidly evolving biological entity is eliminated in the subject.

In some embodiments, the rapidly evolving biological entity is a bacterium. In some embodiments, the bacterium can be any pathogenic bacterium. In some embodiments, the bacterium is of the genus Aspergillus, Brugia, Candida, Chlamydia, Clostridium, Coccidia, Cryptococcus, Dirofilaria, Gonococcus, Enterococcus, Escherichia, Helicobacter, Histoplasma, Leishmania, Mycobacterium, Mycoplasma, Paramecium, Pertussis, Plasmodium, Mycobacterium, Mycoplasma, Pneumococcus, Pneumocystis, Pseudomonas, Rickettsia, Salmonella, Shigella, Staphylococcus, Streptococcus, Toxoplasma or Vibriocholerae. In certain embodiments, the bacterium is of the species Acinetobacter baumannii, Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Microplasma hominis, Mycoplasma adleri, Dermatophilus congolensis, Diplorickettsia massiliensis, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Hemophilus ducreyi, Klebsiella pneumoniae, Granuloma inguinale, Lymphopathia venereum, Treponema pallidum, Mycobacterium tuberculosis, Brucella abortus. Brucella melitensis, Brucella suis, Brucella canis, Campylobacter fetus, Campylobacter fetus intestinalis, Leptospira pomona, Peptostreptococcus anaerobius, Peptostreptococcus asaccharolyticus, Listeria monocytogenes, Staphylococcus aureus, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Streptococcus pneumoniae, Streptococcus pyogenes, Ureaplasma gallorale, Corynebacterium pyogenes, Pasteuria ramosa, Actinobaccilus seminis, Mycoplasma bovigenitalium, Aspergillus fumigatus, Absidia ramosa, Trypanosoma equiperdum, Babesia caballi, Clostridium tetani or Clostridium botulinum.

In some embodiments, the rapidly evolving biological entity is a virus. In some embodiments, the rapidly evolving biological entity can be any virus. In some embodiments, the virus is Human Papilloma Virus (HPV), HBV, hepatitis C Virus (HCV), human immunodeficiency virus (HIV-1, HIV-2), varicella virus, herpes virus, Epstein Barr Virus (EBV), mumps virus, rubella virus, rabies virus, measles virus, viral hepatitis, viral meningitis, cytomegalovirus (CMV), HSV-1, HSV-2, or influenza virus.

The method of any one of claims 1 to 15, wherein the rapidly evolving biological entity is a cancer cell. In some embodiments, the cell can be from any type of cancer, including, but not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the therapeutic nucleic acid is an interfering nucleic acid. In some embodiments, the interfering nucleic acid is an antisense molecule, an siRNA, a single-stranded siRNA or a shRNA. In certain embodiments, the interfering nucleic acid is single stranded. In other embodiments interfering nucleic acid, is double stranded.

In some embodiments, the therapeutic nucleic acid is a nucleic acid aptamer. In one embodiment, the nucleic acid aptamer is an aptamer identified according to one of the aptamer screening methods disclosed herein. In some embodiments, the aptamer is an aptamer from an aptamer library provided herein. In another embodiment, the nucleic acid aptamer is an aptamer of Formula I, II, III, IV or IV.

In certain embodiments, the therapeutic nucleic acid is administered as a pharmaceutical composition, Pharmaceutical compositions described herein include a therapeutic nucleic acid described herein and a pharmaceutically acceptable carrier or vehicle. A pharmaceutical composition described herein is formulated to be compatible with its intended route of administration. In certain embodiments, the pharmaceutical composition is administered via injection (e.g., intravenous injection, intratumoral injection). In some embodiments, the pharmaceutical composition is formulated to be compatible with oral delivery.

### Aptamer Libraries

In certain embodiments, the methods and compositions provided herein relate to the identification of aptamers having desired properties from among the aptamers present in an aptamer library. As used herein, an aptamer library is a collection of nucleic acid molecules (e.g., DNA and/or RNA) having distinct sequences (e.g., at least 10², 10³, 10⁴, 10⁵, 10⁶ or 10⁷ distinct sequences) and wherein at least a subset of the nucleic acid molecules is structured such that they are capable of specifically binding to a target protein or peptide. In some embodiments, any library of potential aptamers can be used in the methods and compositions provided herein.

In some embodiments, the aptamer library used in the methods and compositions provided herein comprises, consists of and/or consists essentially of nucleic acid molecules (e.g., DNA and/or RNA) having a sequence according to Formula (I):

P1-R-P2 (I),

wherein P1 is a 5′ primer site sequence of about 10 to 100 bases in length, about 10 to 50 bases in length, about 10 to 30 bases in length, about 15 to 50 bases in length or about 15 to 30 bases in length; P2 is a 3′ primer site sequence of about 10 to 100 bases in length, about 10 to 50 bases in length, about 10 to 30 bases in length, about 15 to 50 bases in length or about 15 to 30 bases in length; and R is a sequence comprising randomly positioned bases of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length.

In one embodiment, R is a sequence comprising about 25% A. In another embodiment, R is a sequence comprising about 25% T. In another embodiment, R is a sequence comprising about 25% G. In another embodiment, R is a sequence comprising about 25% C. In yet another embodiment, R is a sequence comprising about 25% A, about 25% T, about 25% G, and about 25% C.

In some embodiments, the aptamer library used in the methods and compositions provided herein comprises, consists of and/or consists essentially of nucleic acid molecules (DNA and/or RNA) having a sequence according to Formula (I):

P1-R″-P2 (I),

wherein P1 is a 5′ primer site sequence of about 10 to 100 bases in length, about 10 to 50 bases in length, about 10 to 30 bases in length, about 15 to 50 bases in length or about 15 to 30 bases in length; P2 is a 3′ primer site sequence of about 10 to 100 bases in length, about 10 to 50 bases in length, about 10 to 30 bases in length, about 15 to 50 bases in length or about 15 to 30 bases in length; and R″ is a sequence of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length comprising randomly positioned bases from a biased mixture or any combination of random strings with repetitive or biased strings.

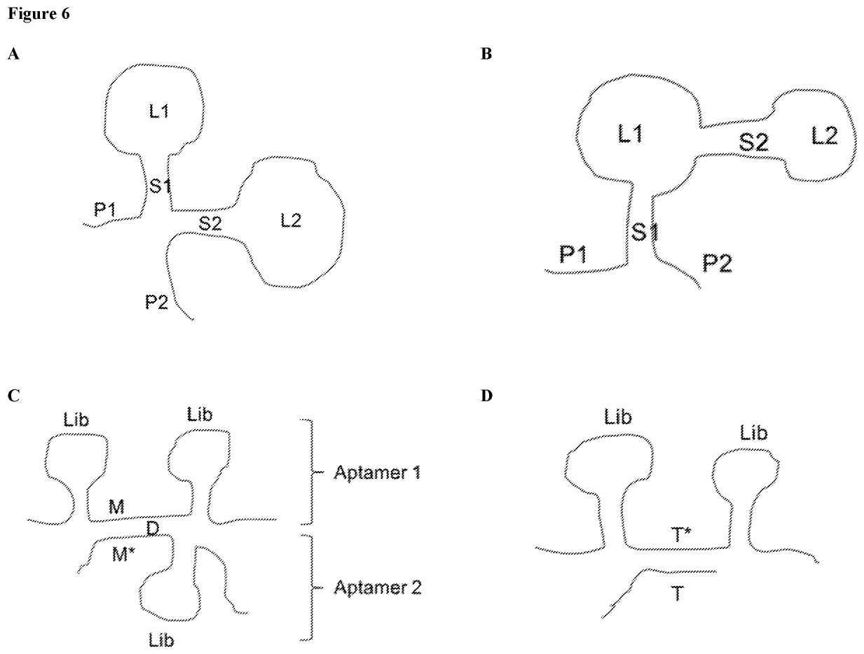

In some embodiments, the aptamer library used in the methods and compositions provided herein comprises, consists of and/or consists essentially of nucleic acid molecules (DNA and/or RNA) having a sequence according to Formula II (an exemplary schematic representation is provided in FIG. 6A),

P1-S1-L1-S1*-S2-L2-S2*-P2 (II),

wherein:

P1 is a 5′ primer site sequence of about 10 to 100 bases in length, about 10 to 50 bases in length, about 10 to 30 bases in length, about 15 to 50 bases in length or about 15 to 30 bases in length; P2 is a 3′ primer site sequence of about 10 to 100 bases in length, about 10 to 50 bases in length, about 10 to 30 bases in length, about 15 to 50 bases in length or about 15 to 30 bases in length; S1 and S2 are each independently a stem region sequence of at least one base (e.g., of about 4 to 40 bases in length or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 bases in length); S1* is a complementary sequence to S1; S2* is a complementary sequence to S2; L1 and L2 are each independently a Loop region sequence of at least one base (e.g., of about 1 to 50 bases in length or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length); and S1-L1-S1*-S2-L2-S2* is collectively about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length.

In some embodiments, the aptamer library used in the methods and compositions provided herein comprises, consists of and/or consists essentially of nucleic acid molecules (DNA and/or RNA) having a sequence according Formula III (an exemplary schematic representation is provided in FIG. 6B):

P1-S1-L1-S2-L2-S2*-L1-S1*-P2 (III),

wherein:

P1 is a 5′ primer site sequence of about 10 to 100 bases in length, about 10 to 50 bases in length, about 10 to 30 bases in length, about 15 to 50 bases in length or about 15 to 30 bases in length; P2 is a 3′ primer site sequence of about 10 to 100 bases in length, about 10 to 50 bases in length, about 10 to 30 bases in length, about 15 to 50 bases in length or about 15 to 30 bases in length;

S1 and S2 are each independently a stem region sequence of at least one base (e.g., of about 4 to 40 bases in length or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 bases in length); S1* is a complementary sequence to S1; S2* is a complementary sequence to S2;

L1 and L2 are each independently a Loop region sequence of at least one base (e.g., of about 1 to 50 bases in length or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length); and

S1-L1-S2-L2-S2*-L1-S1* is collectively about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length.

In some embodiments, the aptamer library used in the methods and compositions provided herein comprises, consists of and/or consists essentially of nucleic acid molecules (DNA and/or RNA) having a sequence according Formula IV (an exemplary schematic representation is provided in FIG. 6C):

P1-Lib-M1/M2-D-M1/M2*-Lib-P2 (IV),

wherein:

P1 is a 5′ primer site sequence of about 10 to 100 bases in length, about 10 to 50 bases in length, about 10 to 30 bases in length, about 15 to 50 bases in length or about 15 to 30 bases in length; P2 is a 3′ primer site sequence of about 10 to 100 bases in length, about 10 to 50 bases in length, about 10 to 30 bases in length, about 15 to 50 bases in length or about 15 to 30 bases in length;

Lib is sequence having a formula selected from: (i) R; (ii) R″; (iii) S1-L1-S1*-S2-L2-S2*; and (iv) S1-L1-S2-L2-S2*-L1-S1*;

R is a sequence comprising randomly positioned bases of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length;

R″ is a sequence of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length comprising randomly positioned bases from a biased mixture or any combination of random strings with repetitive or biased strings; S1 and S2 are each independently a stem region sequence of at least one base (e.g., of about 4 to 40 bases in length or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 bases in length); S1* is a complementary sequence to S1; S2* is a complementary sequence to S2;

L1 and L2 are each independently a Loop region sequence of at least one base (e.g., of about 1 to 50 bases in length or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length);

wherein S1-L1-S1*-S2-L2-S2* is collectively about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length;

D is a spacer sequence comprising at least one base (e.g., of about 1 to 20 bases in length or 1, 2, 3, 4, 5, 6, 7, 8, 9, 10, 11, 12, 13, 14, 15, 16, 17, 18, 19 or 20 bases in length);

M1 is a multimer-forming domain sequence of about 10 to 18 bases in length or 10, 11, 12, 13, 14, 15, 16, 17 or 18 bases in length that enables a strand of the sequence to interact with another strand that contains a complementary domain; and

M2 is a complementary domain of M1 comprising a strand that interacts with a strand of the M1 sequence.

In some embodiments, the aptamer library used in the methods and compositions provided herein comprises, consists of and/or consists essentially of nucleic acid molecules (DNA and/or RNA) having a sequence according Formula V (an exemplary schematic representation is provided in FIG. 6D):

P1-Lib-T*-Lib-P2 (V),

wherein:

P1 is a 5′ primer site sequence of about 10 to 100 bases in length, about 10 to 50 bases in length, about 10 to 30 bases in length, about 15 to 50 bases in length or about 15 to 30 bases in length; P2 is a 3′ primer site sequence of about 10 to 100 bases in length, about 10 to 50 bases in length, about 10 to 30 bases in length, about 15 to 50 bases in length or about 15 to 30 bases in length;

Lib is sequence having a formula selected from: (i) R; (ii) R″; (iii) S1-L1-S1*-S2-L2-S2*; and (iv) S1-L1-S2-L2-S2*-L1-S1*;

R is a sequence comprising randomly positioned bases of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length;

R″ is a sequence of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length comprising randomly positioned bases from a biased mixture or any combination of random strings with repetitive or biased strings;

S1 and S2 are each independently a stem region sequence of at least one base (e.g., of about 4 to 40 bases in length or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 bases in length); S1* is a complementary sequence to S1; S2* is a complementary sequence to S2;

L1 and L2 are each independently a Loop region sequence of at least one base (e.g., of about 1 to 50 bases in length or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length);

wherein S1-L1-S1*-S2-L2-S2* is collectively about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length;

T is a second strand bound by Watson/Crick or Hoogsteen base pairing to any part of the Lib sequence or T*, wherein the strand optionally contains unpaired domains on its 5′ and 3′ ends (e.g., to facilitate attachment of a functional moiety to the aptamer); and

T* is a dedicated domain sequence (e.g., of about 4 to 40 bases in length or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 bases in length).

In some embodiments of the Formulae above, R is randomly positioned bases from any random mixture (e.g., for canonical bases, 25% A, 25% T, 25% G, 25% C) of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length.

In one embodiment of the Formulae above, R is a sequence comprising about 25% A. In another embodiment, R is a sequence comprising about 25% T. In another embodiment, R is a sequence comprising about 25% G. In another embodiment, R is a sequence comprising about 25% C. In yet another embodiment, R is a sequence comprising about 25% A, about 25% T, about 25% G, and about 25% C.

In some embodiments of the Formulae above, R″ is a sequence comprising comprises randomly positioned bases from a biased mixture (e.g., for canonical bases, any mixture deviating from 25% per base). In some embodiments, R″ is a sequence that comprises about 0%, 5%, 10%, 15%, 20%, 25%, 30%, 35%, 40%, 45%, 50%, 55%, 60%, 65%, 70% or 75% A. In some embodiments, R″ is a sequence that comprises about 0%, 5%, 10%, 15%, 20%, 25%, 30%, 35%, 40%, 45%, 50%, 55%, 60%, 65%, 70% or 75% T. In some embodiments, R″ is a sequence that comprises about 0%, 5%, 10%, 15%, 20%, 25%, 30%, 35%, 40%, 45%, 50%, 55%, 60%, 65%, 70% or 75% C. In some embodiments, R″ is a sequence that comprises about 0%, 5%, 10%, 15%, 20%, 25%, 30%, 35%, 40%, 45%, 50%, 55%, 60%, 65%, 70% or 75% G. In some embodiments, R″ is a sequence that comprises any combination of random strings (string is any sequence including a single base) with repetitive or biased strings.

In some embodiments of the Formulae above, R″ is randomly positioned bases from a biased mixture (e.g., for canonical bases, any mixture deviating from 25% per base); or any combination of random strings (string is any sequence including a single base) with repetitive or biased strings of about at least 10, 15, 20, 25, 30, 35, 40, 45, 50, 55, 60, 65, 70, 75 or 80 bases in length and/or no more than about 120, 115, 110, 105, 100, 95, 90, 85, 80, 75, 70, 65, 60, 55 or 50 bases in length.

In some embodiments of the Formulae above, S1 is a stem region sequence of at least 1 base or more. In other embodiments, S1 is a stem region sequence of between about 4 to 40 bases in length or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 bases in length.

In some embodiments of the Formulae above, S2 is a stem region sequence of at least 1 base or more. In other embodiments, S2 is a stem region sequence of between about 4 to 40 bases in length or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 bases in length.

In some embodiments of the Formulae above, L1 is a Loop region sequence of at least one base. In other embodiments, L1 is a Loop region sequence of about 1 to 50 bases in length or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length.

In some embodiments of the Formulae above, L2 is a Loop region sequence of at least one base. In other embodiments, L2 is a Loop region sequence of about 1 to 50 bases in length or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length.

In some embodiments of the Formulae above, T may include unpaired domains on its 5′ and 3′ ends, or it may be a padlock tail (e.g., a loop between two domains paired with the library).

The aptamers of the present disclosure may contain any number of stems and loops, and other structures comprised of stems and loops (e.g., hairpins, bulges, etc.). In some embodiments, the loops in the aptamer contain bases implanted in order to form stable loop-loop WC pairing forming a stem which is orthogonal to the main library axis. In other embodiments, two loops in the aptamer together form an orthogonal stem. In yet other embodiments, the loops in the aptamer contain bases implanted in order to form stable Hoogsteen pairing with an existing stem along the main library axis. In other embodiments, the loops in the aptamer can form Hoogsteen pairing with any stem in the aptamer.

In some embodiments of the formulae above, the aptamer sequence further contains one or more multimer-forming domains.

In some embodiments of the formulae above, the aptamer sequence further contains one or more spacers (e.g., of about 1 to 20 bases in length or 1, 2, 3, 4, 5, 6, 7, 8, 9, 10, 11, 12, 13, 14, 15, 16, 17, 18, 19 or 20 bases in length).

The aptamers of the present disclosure can be prepared in a variety of ways. In one embodiment, the aptamers are prepared through chemical synthesis. In another embodiment, the aptamers are prepared through enzymatic synthesis. In one embodiment, the enzymatic synthesis can be carried out using any enzyme that can add nucleotides to elongate a primer, with or without template. In some embodiments, the aptamers are prepared by assembling together k-mers (e.g., k≥2 bases).

In some embodiments, the aptamers of the present disclosure may contain any combination of DNA, RNA, and their natural and/or synthetic analogs. In one embodiment, the aptamer comprises DNA. In one embodiment, the aptamer comprises RNA.

In other embodiments, the aptamers of the present disclosure may contain any modification on the 5′ end, 3′ end, or internally. Modifications of the aptamers include, but are not limited to, spacers, phosphorylation, linkers, conjugation chemistries, fluorophores, quenchers, photoreactive, and modified bases (e.g., LNA, PNA, UNA, PS, methylation, 2-O-methyl, halogenated, superbases, iso-dN, inverted bases, L-ribose, other sugars as backbone, etc.).

In some embodiments, the aptamers of the present disclosure may be conjugated to external, non-nucleic acid molecules on the 5′ end, 3′ end, or internally. Non-limiting examples of non-nucleic acid molecules include, but are not limited to. amino acids, peptides, proteins, small molecule drugs, mono- and polysaccharides, lipids, antibodies and antibody fragments, or a combination thereof.

The aptamers of the present disclosure may contain any domain which has a biological function. Non-limiting examples of biological functions of the aptamers described herein include, but are not limited to, acting as templates for RNA transcription, binding to, recognizing, and/or modulating the activity of proteins, binding to transcription factors, specialized nucleic acid structure (e.g., Z-DNA, H-DNA, G-quad, etc.), and acting as an enzymatic substrate for restriction enzymes, specific exo- and endonucleases, recombination sites, editing sites, or siRNA. In one embodiment, the aptamers modulate the activity of at least one protein. In another embodiment, the aptamers inhibit the activity of at least one protein. In yet another embodiment, the aptamers inhibit the activity of at least one protein

In other embodiments, the aptamers of the present disclosure may contain any domain for integration into a nucleic acid nanostructure built by any one of several known methods (Shih et al, Nature 427:618-621 (2004); Rothemund, Nature 440:297-302 (2006); Zheng et al, Nature 461:74-77 (2009); Dietz et al, Science 325:725-730 (2009); Wei et al, Nature 485:623-626 (2012); Ke et al, Science 338:1177-1183 (2012); Douglas et al, Science 335:831-834 (2012), each of which are hereby incorporated by reference). In yet other embodiments, the aptamers of the present disclosure may contain any domain that serves a function in molecular logic and computation (Seelig et al, Science 314:1585-1588 (2006); Macdonald et al, Nano Lett 6:2598-2603 (2006); Qian et al, Nature 475:368-372 (2011); Douglas et al, Science 335:831-834 (2012); Amir et al, Nat Nanotechnol 9:353-357 (2014), each of which is hereby incorporated by reference).

In some embodiments, the aptamers of the present disclosure undergo one or more cycles of negative selection versus a target (e.g., eukaryotic or prokaryotic cell, virus or viral particle, molecule, tissue, or whole organism, in-vivo or ex-vivo). In other embodiments, the aptamers of the present disclosure undergo one or more cycles of positive selection versus a target (e.g., eukaryotic or prokaryotic cell, virus or viral particle, molecule, tissue, or whole organism, in-vivo or ex-vivo).

The aptamers of the present disclosure can be in solution or attached to a solid phase (e.g., surface, particles, resin, matrix, etc.). In some embodiments, the aptamer is attached to a surface. In one embodiment, the surface is a flow cell surface.

In some embodiments, the aptamers of the present disclosure are synthesized in an aptamer library. The aptamer library of the present disclosure can be prepared in a variety of ways. In one embodiment, the aptamer library is prepared through chemical synthesis. In another embodiment, the aptamer library is prepared through enzymatic synthesis. In one embodiment, the enzymatic synthesis can be carried out using any enzyme that can add nucleotides to elongate a primer, with or without template.

In some embodiments, the aptamers synthesized in an aptamer library may contain any combination of DNA, RNA, and their natural and/or synthetic analogs. In one embodiment, the aptamers synthesized in an aptamer library comprise DNA. In one embodiment, the aptamers synthesized in an aptamer library comprise RNA.

In some embodiments, the aptamers synthesized in an aptamer library are a nucleic acid (e.g., DNA, RNA, natural or synthetic bases, base analogs, or a combination thereof) collection of 10^{K} species (K≥2), with Z copies per species (1≤Z≤K−1).

In other embodiments, the aptamers synthesized in an aptamer library of the present disclosure may contain any modification on the 5′ end, 3′ end, or internally. Modifications of the aptamers include, but are not limited to, spacers, phosphorylation, linkers, conjugation chemistries, fluorophores, quenchers, photoreactive modifications, and modified bases (e.g., LNA, PNA, UNA, PS, methylation, 2-O-methyl, halogenated, superbases, iso-dN, inverted bases, L-ribose, other sugars as backbone).

In some embodiments, the aptamers synthesized in an aptamer library may be conjugated to external, non-nucleic acid molecules on the 5′ end, 3′ end, or internally. Non-limiting examples of non-nucleic acid molecules include, but are not limited to. amino acids, peptides, proteins, small molecule drugs, mono- and polysaccharides, lipids, antibodies and antibody fragments, or a combination thereof.

The aptamers synthesized in an aptamer library may contain any domain which has a biological function. Non-limiting examples of biological functions of the aptamers described herein include, but are not limited to, acting as templates for RNA transcription, binding to, recognizing, and/or modulating the activity of proteins, binding to transcription factors, specialized nucleic acid structure (e.g., Z-DNA, H-DNA, G-quad, etc.), acting as an enzymatic substrate for restriction enzymes, specific exo- and endonucleases, recombination sites, editing sites, or siRNA. In one embodiment, the aptamers synthesized in an aptamer library modulate the activity of at least one protein. In another embodiment, the aptamers synthesized in an aptamer library inhibit the activity of at least one protein. In yet another embodiment, the aptamers synthesized in an aptamer library inhibit the activity of at least one protein

In other embodiments, the aptamers synthesized in an aptamer library may contain any domain for integration into a nucleic acid nanostructure built by one of several known methods (Shih et al, Nature 427:618-621 (2004); Rothemund, Nature 440:297-302 (2006); Zheng et al, Nature 461:74-77 (2009); Dietz et al, Science 325:725-730 (2009); Wei et al, Nature 485:623-626 (2012); Ke et al, Science 338:1177-1183 (2012); Douglas et al, Science 335:831-834 (2012), each of which are hereby incorporated by reference). In yet other embodiments, the aptamers of the present disclosure may contain any domain that serves a function in molecular logic and computation (Seelig et al, Science 314:1585-1588 (2006); Macdonald et al, Nano Lett 6:2598-2603 (2006); Qian et al, Nature 475:368-372 (2011); Douglas et al, Science 335:831-834 (2012); Amir et al, Nat Nanotechnol 9:353-357 (2014), each of which is hereby incorporated by reference)

In some embodiments, the aptamers synthesized in an aptamer library undergo one or more cycles of negative selection versus a target (e.g., eukaryotic or prokaryotic cell, virus or viral particle, molecule, tissue, or whole organism, in-vivo or ex-vivo). In other embodiments, the aptamers of the present disclosure undergo one or more cycles of positive selection versus a target (e.g., eukaryotic or prokaryotic cell, virus or viral particle, molecule, tissue, or whole organism, in-vivo or ex-vivo).

The aptamers synthesized in an aptamer library can be in solution or attached to a solid phase (e.g., surface, particles, resin, matrix, etc.). In some embodiments, the aptamers synthesized in an aptamer library are attached to a surface. In one embodiment, the surface is a flow cell surface.

### Immobilized Aptamer Clusters

In certain aspects, provided herein are methods for identifying aptamers that bind to and/or modulate a rapidly evolving biological target by flowing a sample comprising the target across a plurality of aptamer clusters (e.g., clusters of aptamers from the aptamer libraries provided herein) immobilized on a surface. In certain embodiments the surface can be any solid support. In some embodiments, the surface is the surface of a flow cell. In some embodiments, the surface is a slide or chip (e.g., the surface of a gene chip). In some embodiments, the surface is a bead (e.g., a paramagnetic bead).

In certain embodiments, any method known in the art can be used to generate the immobilized aptamer clusters on the surface. In some embodiments, the aptamer clusters are printed directly onto the surface. For example, in some embodiments, the aptamer clusters are printed with fine-pointed pins onto glass slides, printed using photolithography, printed using ink-jet printing, or printed by electrochemistry on microelectrode arrays. In some embodiments, at least about 10², 10³, 10⁴, 10⁵, 10⁶ or 10⁷ distinct aptamer clusters are printed onto the surface. In some embodiments, each aptamer cluster comprises at least about 10, 20, 30, 40, 50, 60, 70, 80, 90, 100, 125, 150, 175, 200, 250, 300, 350, 400, 450, 500, 600, 700, 800, 900, 1000, 2000, 3000, 4000, 5000, 10,000, 20,000, 30,000, 40,000, 50,000, 60,000, 70,000, 80,000, 90,000 or 100,000 identical aptamer molecules. Advantageously, direct printing of microarrays allows for aptamers of known sequence to be specifically immobilized at a predetermined position on the surface, so subsequent sequencing may be unnecessary.

In certain embodiments, the surface-immobilized aptamer clusters are generated by first immobilizing aptamers (e.g., from an aptamer library disclosed herein) onto the surface (e.g., wherein the position at which each aptamer is immobilized is random). In some embodiments, at least about 10², 10³, 10⁴, 10⁵, 10⁶, 10⁷, 10⁸, 10⁹ or 10¹⁰ distinct aptamers are immobilized onto the surface. Following aptamer immobilization, a localized amplification process (e.g., bridge amplification or rolling circle amplification), is then performed to generate clusters of copies of each immobilized aptamer positioned proximal to the immobilization site of the original immobilized aptamer. In certain embodiments (e.g., embodiments in which rolling circle amplification is performed) the aptamer cluster is housed in a nano-pit or pore on the surface rather than being directly immobilized on the surface. In some embodiments, amplification results in each aptamer cluster comprising at least about 10, 20, 30, 40, 50, 60, 70, 80, 90, 100, 125, 150, 175, 200, 250, 300, 350, 400, 450, 500, 600, 700, 800, 900, 1000, 2000, 3000, 4000, 5000, 10,000, 20,000, 30,000, 40,000, 50,000, 60,000, 70,000, 80,000, 90,000 or 100,000 identical aptamer molecules. In certain embodiments, the aptamer clusters are then sequenced (e.g., by IIlumina sequencing or Polonator sequencing) in order to associate the sequence of each aptamer cluster with its position on the surface. If present, complementary strands can be stripped from the aptamer cluster by washing the surface under conditions not amenable to strand hybridization (e.g., due to salt concentration and/or temperature) in order to generate clusters of single-stranded aptamers. The surface (e.g., flow cell) is then ready for use in an aptamer identification method provided herein. In some embodiments, the immobilized aptamer clusters are prepared and/or sequenced on one instrument, and then transferred to a separate instrument for aptamer identification. In other embodiments, the aptamer clusters are prepared and/or sequenced on the same instrument as is used for aptamer identification.

In some embodiments of the methods above, the aptamers or aptamer clusters (e.g., from the aptamer library) comprise an adapter that will bring the aptamers to surface height (e.g., in cases where the surface is not flat, such as in flow cells that include pores). In one embodiment, the aptamers or aptamer clusters are immobilized inside pores on a flow cell surface and adapters are used to bind the aptamer to the surface in order to bring the aptamers to surface height. In some embodiments, the adapter is a nucleic acid adapter (e.g., a sequence of at least about 10, 15, 20, 25, 30, 35, 40, 45, 50, 55, 60, 65, 70, 75, 80, 85, 90, 95 or 100 bases in length). In some embodiments, a sequence complementary to the adapter sequence is hybridized to the adapter prior to aptamer screening. In some embodiments, the adapter is a chemical adapter (e.g., a polymer connecting the aptamer to the surface).

### Aptamer Library Screening

In certain aspects, provided herein include screening assays for identifying one or more aptamers that specifically bind to and/or modulate a target (e.g., a rapidly-evolving target), the method generally comprising: (i) contacting a plurality of aptamer clusters immobilized on a surface with the target; and (ii) identifying the immobilized aptamer clusters that specifically bind to and/or modulate the target. Because the sequence of each aptamer cluster is associated with a specific position on the surface (e.g., determined according to the methods provided herein), the sequence of the aptamer responsible for the binding/modulation is identified and the position at which the target is bound and/or modulated can be determined.

In some embodiments, the target is labeled with and/or comprises a detectable label. The target can be detectably labeled directly (e.g., through a direct chemical linker) or indirectly (e.g., using a detectably labeled target-specific antibody). In embodiments in which the target is a cell, it can be labeled by incubating the target cell with the detectable label under conditions such that the detectable label is internalized by the cell. In some embodiments, the target is detectably labeled before performing the aptamer screening methods described herein. In some embodiments, the target is labeled during the performance of the aptamer screening methods provided herein. In some embodiments, the target is labeled after is it is bound to an aptamer cluster (e.g., by contacting the bound target with a detectably labeled antibody). In some embodiments, any detectable label can be used. Examples of detectable labels include, but are not limited to, fluorescent moieties, radioactive moieties, paramagnetic moieties, luminescent moieties and/or colorimetric moieties. In some embodiments, the targets described herein are linked to, comprise and/or are bound by a fluorescent moiety. Examples of fluorescent moieties include, but are not limited to, Allophycocyanin, Fluorescein, Phycoerythrin, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet.

The target can be a non-molecular or a supramolecular target. Non-limiting examples of targets to which the aptamers of the present disclosure can bind to and/or modulate include, but are not limited to, cells, bacteria, fungi, archaea, protozoa, viruses, virion particles, synthetic and naturally-occurring microscopic particles, and liposomes. In some embodiments, the target introduced into the flow cell is live/native. In other embodiments, the target introduced into the flow cell is fixed in any solution.

In some embodiments, the target is a cell. In some embodiments, the cell is a prokaryotic cell. In some embodiments, the cell is a bacterial cell. In other embodiments, the bacteria is a gram-positive bacterium. In yet other embodiments, the bacteria is a gram-negative bacterium. Non-limiting examples of bacteria include Acinetobacter baumannii, Aspergillus, Anaerococcus, Brugia, Candida, Chlamydia (Genus), Clostridium, Coccidia, Cryptococcus, Dermatophilus congolensis, Diplorickettsia massiliensis, Dirofilaria, Enterococcus, Escherichia, Gonococcus, Helicobacter, Histoplasma, Klebsiella, Mycoplasma, Legionella, Leishmania, MafB toxins, Meningococci, Mobiluncus, Mycobacterium, Mycoplasma, Neisseria, Pasteuria, Paramecium, Pathogenic bacteria, Peptostreptococcus, Pertussis, Plasmodium, Pneumococcus, Pneumocystis, Pseudomonas, Rickettsia, Salmonella, Shigella, Staphylococcus, Streptococcus, Toxoplasma and Vibriocholerae. Exemplary species include Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Streptococcus pneumoniae, Streptococcus pyogenes, Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma adleri, Mycoplasma bovigenitalium, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Pasteuria ramosa, Peptostreptococcus anaerobius, Peptostreptococcus asaccharolyticus, Pontiac fever, Aspergillus fumigatus, Absidia ramosa, Staphylococcus aureus, Trypanosoma equiperdum, Ureaplasma gallorale, Klebsiella pneumonia, Babesia caballi, Clostridium tetani, and Clostridium botulinum. In some embodiments, the cell is a eukaryotic cell. In some embodiments, the cell is an animal cell (e.g., a mammalian cell). In some embodiments, the cell is a human cell. In some embodiments, the cell is from a non-human animal, such as a mouse, rat, rabbit, pig, bovine (e.g., cow, bull, buffalo), deer, sheep, goat, llama, chicken, cat, dog, ferret, or primate (e.g., marmoset, rhesus monkey). In some embodiments, the cell is a parasite cell (e.g., a malaria cell, a leishmanias cell, a cryptosporidium cell or an amoeba cell). In some embodiments, the cell is a fungal cell, such as, e.g., Paracoccidioides brasiliensis.

In some embodiments, the cell is a cancer cell (e.g., a human cancer cell). In some embodiments, the cell is from any cancerous or pre-cancerous tumor. Non-limiting examples of cancer cells include cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant, carcinoma, carcinoma, undifferentiated, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma, gastrinoma, malignant, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, malignant, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, paget's disease, mammary, acinar cell carcinoma, adenosquamous carcinoma, adenocarcinoma w/squamous metaplasia, thymoma, malignant, ovarian stromal tumor, malignant, thecoma, malignant, granulosa cell tumor, malignant, and roblastoma, malignant, sertoli cell carcinoma, leydig cell tumor, malignant, lipid cell tumor, malignant, paraganglioma, malignant, extra-mammary paraganglioma, malignant, pheochromocytoma, glomangiosarcoma, malignant melanoma, amelanotic melanoma, superficial spreading melanoma, malig melanoma in giant pigmented nevus, epithelioid cell melanoma, blue nevus, malignant, sarcoma, fibrosarcoma, fibrous histiocytoma, malignant, myxosarcoma, liposarcoma, leiomyosarcoma, rhabdomyosarcoma, embryonal rhabdomyosarcoma, alveolar rhabdomyosarcoma, stromal sarcoma, mixed tumor, malignant, mullerian mixed tumor, nephroblastoma, hepatoblastoma, carcinosarcoma, mesenchymoma, malignant, brenner tumor, malignant, phyllodes tumor, malignant, synovial sarcoma, mesothelioma, malignant, dysgerminoma, embryonal carcinoma, teratoma, malignant, struma ovarii, malignant, choriocarcinoma, mesonephroma, malignant, hemangiosarcoma, hemangioendothelioma, malignant, kaposi's sarcoma, hemangiopericytoma, malignant, lymphangiosarcoma, osteosarcoma, juxtacortical osteosarcoma, chondrosarcoma, chondroblastoma, malignant, mesenchymal chondrosarcoma, giant cell tumor of bone, ewing's sarcoma, odontogenic tumor, malignant, ameloblastic odontosarcoma, ameloblastoma, malignant, ameloblastic fibrosarcoma, pinealoma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, malignant, neurofibrosarcoma, neurilemmoma, malignant, granular cell tumor, malignant, malignant lymphoma, Hodgkin's disease, Hodgkin's lymphoma, paragranuloma, malignant lymphoma, small lymphocytic, malignant lymphoma, large cell, diffuse, malignant lymphoma, follicular, mycosis fungoides, other specified non-Hodgkin's lymphomas, malignant histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, and hairy cell leukemia.

The therapeutic nucleic acids (e.g., aptamers) of the present disclosure can be directly cytotoxic (e.g., inducing apoptosis through a cellular mechanism, catalytically/mechanically perturbing target structural integrity, etc.), indirectly cytotoxic (inducing a host defense response against the target, etc.), growth-inhibiting, or recognition/binding-neutralizers (in the case of viruses or other pathogens binding to cells in order to enter them, etc.).

In some embodiments, the target is a virus. For example, in some embodiments, the virus is HIV, hepatitis A, hepatitis B, hepatitis C, herpes virus (e.g., HSV-1, HSV-2, CMV, HAV-6, VZV, Epstein Barr virus), adenovirus, influenza virus, flavivirus, echovirus, rhinovirus, coxsackie virus, coronavirus, respiratory syncytial virus, mumps virus, rotavirus, measles virus, rubella virus, parvovirus, vaccinia virus, HTLV, dengue virus, papillomavirus, molluscum virus, poliovirus, rabies virus, JC virus, Human papilloma virus (HPV), Infectious mononucleosis, viral gastroenteritis (stomach flu), viral hepatitis, viral meningitis, viral pneumonia, rabies virus, or ebola virus.

In some embodiments, the property of the cell that is modulated is cell viability, cell proliferation, gene expression, cellular morphology, cellular activation, phosphorylation, calcium mobilization, degranulation, cellular migration, and/or cellular differentiation. In certain embodiments, the target is linked to, bound by or comprises a detectable label that allows for the detection of a biological or chemical effect on the target. In some embodiments, the detectable label is a fluorescent dye. Non-limiting examples of fluorescent dyes include, but are not limited to, a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, and a redox potential dye. In one embodiment, the target is labeled with a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, or a redox potential dye.

In certain embodiments, the target is labeled with an activation associated marker, an oxidative stress reporter, an angiogenesis marker, an apoptosis marker, an autophagy marker, a cell viability marker, or a marker for ion concentrations. In yet another embodiment, the target is labeled with an activation associated marker, an oxidative stress reporter, an angiogenesis marker, an apoptosis marker, an autophagy marker, a cell viability marker, or a marker for ion concentrations prior to exposure of aptamers to the target.

In some embodiments, the target is labeled after to exposure of aptamers to the target. In one embodiment, the target is labeled with fluorescently-labeled antibodies, annexin V, antibody fragments and artificial antibody-based constructs, fusion proteins, sugars, or lectins. In another embodiment, the target is labeled with fluorescently-labeled antibodies, annexin V, antibody fragments and artificial antibody-based constructs, fusion proteins, sugars, or lectins after exposure of aptamers to the target.

In some embodiments, the target cell is labeled with a fluorescent dye. Non-limiting examples of fluorescent dyes include, but are not limited to, a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, and a redox potential dye.

In some embodiments, the target cell is labeled with a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, or a redox potential dye. In certain embodiments, the target cell is labeled with an activation associated marker, an oxidative stress reporter, an angiogenesis marker, an apoptosis marker, an autophagy marker, a cell viability marker, or a marker for ion concentrations. In yet another embodiment, target cell is labeled with an activation associated marker, an oxidative stress reporter, an angiogenesis marker, an apoptosis marker, an autophagy marker, a cell viability marker, or a marker for ion concentrations prior to exposure of aptamers to the cell. In some embodiments, the target cell is labeled after to exposure of aptamers to the target. In one embodiment, the target cell is labeled with a fluorescently-labeled antibody or antigen-binding fragment thereof, annexin V, a fluorescently-labeled fusion protein, a fluorescently-labeled sugar, or fluorescently labeled lectin. In one embodiment, the target cell is labeled with a fluorescently-labeled antibody or antigen-binding fragment thereof, annexin V, a fluorescently-labeled fusion protein, a fluorescently-labeled sugar, or fluorescently labeled lectin after exposure of aptamers to the cell.

The position of the detectable marker on the surface can be determined using any method known in the art, including, for example, fluorescent microscopy.

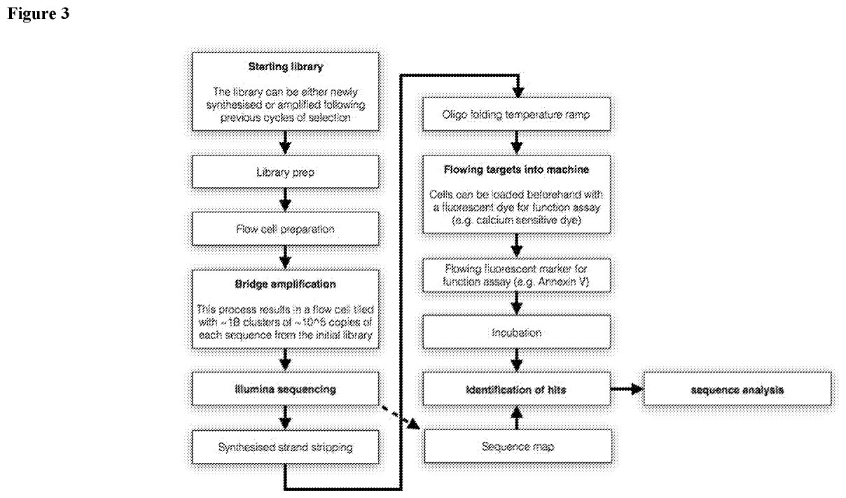

FIG. 3 provides an exemplary workflow illustrating certain embodiments of the methods provided herein. The workflow begins with an initial aptamer library (e.g., an aptamer library provided herein) chosen and prepared as though for Illumina sequencing. The library can be, for example, newly synthesized, or an output of a previous selection process. This process can involve one or more positive selection cycles, one or more negative selection cycles, or both, in either combination and sequence.

The prepared library is mounted on adapters on an Illumina flow cell. Bridge PCR amplification turns each single sequence from the initial library into a cluster of about 100,000 copies of the same sequence. The library is then Illumina-sequenced. This process produces a map linking each sequence from the library to a specific set of coordinates on the flow cell surface.

The complementary strands to those from the library, added in the process of sequencing by synthesis, are stripped by any one of a number of methods (e.g., detergents, denaturing agents, etc.). The oligonucleotide strands complementary to the Illumina adapter and to the PCR primers are then pumped into the flow cell, leaving only the aptamer region single-stranded. When RNA aptamers are being synthesized as part of the library, transcription is initiated and halted by any one of a number of methods (e.g., Ter-bound Tus protein, or biotin-bound streptavidin protein).

The flow cell temperature is raised and then cooled, in order to allow all oligonucleotides on the surface to assume their proper 3D structure, folding according to a folding protocol. In this state, the oligo library is folded and ready to engage targets.

The solution comprising the targets is run into the flow cell using the instrument's hardware. The targets can be labeled prior to introduction into the flow cell/instrument with a fluorescent dye, for the purpose of reporting a biological or chemical effect on the target. The targets are incubated for a certain amount of time to allow the effect to take place. Fluorescent dyes or markers for reporting the biological or chemical effect (e.g., cell activation, apoptosis, etc.) can then be pumped into the flow cell. (See FIG. 3) Affected targets (hits) are recognized by image analysis, and corresponding sequences are analyzed. Extracted sequences are synthesized and tested separately for binding and function.

### EXAMPLES

### Example 1—Preparation of Aptamer Library

Aptamer libraries were prepared using an Illumina high throughput sequencing platform sample preparation kits which included the attachment of an adapter DNA sequence on the flanks of the sample sequence to complement strands already attached to the surface of the flow cell. The prepared library was mounted onto adapters on the surface of an Illumina flow cell.

For the preparation of the aptamer libraries, a two-step “tail” PCR process was used to attach the adapters. The PCR reaction mix contained the following components shown in Table 1:

TABLE 1

| Component | Amount in μl |
|---|---|
| Herculase II fusion DNA polymerase | 0.5 |
| buffer | 10 |
| Dntp (10 mM each) | 1.25 |
| Forward tail primer | 1 |
| Reverse tail primer | 1 |
| upw | 35.25 |
| sample | 1 |

The primers were set in a way that adapters would have a specific orientation with respect to the sample sequence. This was done to hold the forward aptamer sequence in the clusters in a single read run.

The sequence of the primers used in 1st PCR reaction:

TruSeq p7 side start
[SEQ ID NO: 3]
GTCACATCTCGTATGCCG TCTTCTGCTTG ATCCAGAGT
GACGCAGCA;
and
TruSeq p5 side stab reverse primer
[SEQ ID NO: 2]
CTCTTTCCCTACACGACG CTCTTCCGATCT ACTAAGCC
ACCGTGTCCA

The PCR program used for the first reaction is shown herein below in Table 2:

TABLE 2

| Step | Temperature | Time (seconds) |
|---|---|---|
| 1 | 95 | 180 |
| 2 | 95 | 30 |
| 3 | 56 | 10 |
| 4 | 72 | 10 |
| 5 | Return to step 2 × 3 | |
| 6 | 95 | 30 |
| 7 | 85 | 10 |
| 8 | 72 | 10 |
| 9 | Return to step 6 × 10 | |
| 10 | 4 | Forever |

The product of first PCR reaction (PCR 1) is the input for the 2nd PCR reaction.

The sequence of the primers used in the 2nd PCR reaction:

TruSeq p7 side start
[SEQ ID NO: 3]
GATCGGAAGAGCACACGTCTGAACTC
CAGTCACATCTCGTATGCCG;
and
TruSeq p5 side start
[SEQ ID NO: 4]
AATGATACGGCGACCACCGAGATCTA
CACACACTCTTTCCCTACACGACG.

The PCR program used for the second reaction is shown herein below in Table 3:

TABLE 3

| Step | Temperature | time |
|---|---|---|
| 1 | 95 | 30 |
| 2 | 67 | 10 |
| 3 | 72 | 10 |
| 4 | 95 | 30 |
| 5 | 65 | 10 |
| 6 | 72 | 10 |
| 7 | 95 | 30 |
| 8 | 63 | 10 |
| 9 | 72 | 10 |
| 10 | 95 | 30 |
| 11 | 62 | 10 |
| 12 | 72 | 10 |
| 13 | 95 | 30 |
| 14 | 87 | 10 |
| 15 | 72 | 10 |
| 16 | Return to step 13 × 1 | |
| 17 | 95 | 30 |
| 18 | 85 | 10 |
| 19 | 72 | 10 |
| 20 | Return to step 17 × 7 | |
| 21 | 4 | Forever |

Completed libraries underwent quality control which included qbit check for concentration and tapstation/fragment analyzer to check for library size and byproducts. Cluster generation and sequencing was carried out according to the sequencing platform and Illumina protocols. After the sequencing process, denaturation provides the clusters in a single strand form. Adapters and primers are then blocked and aptamers will fold to their 3d conformation in their folding buffer.

### Generation and Sequencing of Clusters

Bridge PCR amplification was used to turn each single sequence from the initial library into a cluster of about 100,000 copies of the same sequence. The cluster library was then Illumina-sequenced. This process produced a map linking each sequence from the library to a specific set of coordinates on the flow cell surface.

The complementary strands to those from the library, added in the process of sequencing by synthesis, were stripped and oligonucleotide strands complementary to the Illumina adapter and to the PCR primers were pumped to the flow cell, leaving only the aptamer region single-stranded. In case of RNA aptamers, transcription was initiated and halted by any one of a number of methods (e.g., Ter-bound Tus protein, or biotin-bound streptavidin protein).

The flow cell temperature was raised and then cooled, to allow all oligonucleotides on the surface of the flow cell to assume their proper 3D conformation in the appropriate folding buffer. For example, one folding buffer recipe used (cellselex paper) included 1 liter PBS, 5 ml of 1M MgCl₂, and 4.5 g glucose

### Target Introduction

Target (e.g., cells, bacteria, particles, viruses, proteins, etc.) were introduced into the system in the desired binding buffer according to the environment they would be used in (e.g., human serum, PBS, lb) using the machine's hardware. One option for a general binding buffer recipe is (cellselsex paper): 1 liter PBS, 5 ml 1M MgCl₂, 4.5 g glucose, 100 mg tRNA, and 1 g BSA. Targets were labeled prior to or after introduction into the flow cell/machine and incubated for a certain amount of time to let effect take place.

Targets can be labeled using different fluorophore that will fit the platforms excitation source and emission filters. Labeling can be done through any possible docking site available on the target. Examples of labeling agents include, but are not limited to, DiI, anti HLA+secondary Dylight 650, anti HLA PE-Cy5, and Dylight 650.

For the screening of functional aptamers, fluorescent reporters can be used to visualize the effect. For example, introduction of 7AAD to the flow cell can be used to label the targets to screen for cell death, or annexin V fluorophore conjugate can be used to label the targets to screen for apoptosis. The reporter agent, its concentration, time of incubation and specific recipe protocol should be adjusted in accordance with the specific effect screening for.

### Representative Method for Sequencing Initial Library Followed by Target Cell Introduction and Acquisition of Functional Oligonucleotide Clusters

80 μl of “Incorporation Mix Buffer” is pumped into the flow cell at a rate of 250 μl/min. The temperature is then set temperature to 55° C. 60 μl of “Incorporation Mix” is pumped to the flow cell at a rate of 250 μl/min and after 80 seconds 10 μl of “Incorporation Mix” is pumped to the flow cell at a rate of 250 μl/min. After 211 seconds, the temperature is set to 22° C. and 60 μl of “Incorporation Mix Buffer” is pumped to the flow cell at a rate of 250 μl/min. 75 μl of “Scan Mix” is then pumped into to the flow cell at a rate of 250 μl/min.

The method then calibrates to focus to the plane of the clusters and align microscope and flow cell planes. 100 μl of “Incorporation Mix Buffer” is pumped into to the flow cell at a rate of 250 μl/min. The incorporation steps above are repeated 99 times.

The temperature control is turned off and 125 μl of “Cleavage Buffer” is pumped into the flow cell at a rate of 250 μl/min. The temperature is then set to 55° C. and 75 μl of “Cleavage Mix” pumped into the to the flow cell at a rate of 250 μl/min. After 80 seconds, 25 μl of “Cleavage Mix” is pumped into the flow cell at a rate of 250 μl/min. After an addition 80 seconds, 25 μl of “Cleavage Mix” is pumped into the flow cell at a rate of 250 μl/min. After 80 seconds, the temperature is set to 22° C. The temperature control is then turned off and 60 μl of “Incorporation Mix Buffer” is pumped into the flow cell at a rate of 250 μl/min. The volume remaining in each water tube is then checked to verify proper delivery.

Denaturation then takes place followed by capping. For the denaturation steps, the temperature is then set to 20° C. for 120 seconds. 75 μl of “Wash Buffer” is pumped into the flow cell at a rate of 60 μl/min, followed by 75 μl of “Denaturation Solution” at a rate of 60 μl/min and 75 μl of “Wash Buffer” at a rate of 60 μl/min.

For the capping steps, 75 μl of “Wash Buffer” is pumped into the flow cell at a rate of 60 μl/min and the temperature is set to 85° C. for 120 seconds. 80 μl of “5′ Cap” is then pumped into the flow cell at a rate of 80 μl/min and the temperature is set to 85° C. for 30 seconds. 10 μl of “5′ Cap” is pumped into the flow cell at a rate of 13 μl/min and the temperature is set to 85° C. for 60 seconds. 10 μl of “5′ Cap” is pumped into the flow cell at a rate of 13 μl/min and the temperature is set to 85° C. for 90 seconds. 10 μl of “5′ Cap” is pumped into to the flow cell at a rate of 13 μl/min and the temperature is set to 85° C. for 120 seconds. 10 μl of “5′ Cap” is pumped into the flow cell at a rate of 13 μl/min and the temperature is set to 85° C. for 150 seconds.

10 μl of “5′ Cap” is pumped into the flow cell at a rate of 13 μl/min and the temperature is set to 85° C. for 180 seconds. 10 μl of “5′ Cap” is pumped into the flow cell at a rate of 13 μl/min and the temperature is set to 85° C. for 210 seconds. 10 μl of “5′ Cap” is pumped into the flow cell at a rate of 13 μl/min and the temperature is set to 85° C. for 240 seconds. 10 μl of “5′ Cap” is pumped into the flow cell at a rate of 13 μl/min and the temperature is set to 85° C. for 270 seconds. 75 μl of “Wash Buffer” is pumped into the flow cell at a rate of 60 μl/min and the temperature is set to 85° C. for 120 seconds.

For the 3′ Cap, 80 μl of “3′ Cap” is pumped into the flow cell at a rate of 80 μl/min and the temperature is set to 85° C. for 30 seconds. 10 μl of “3′ Cap” is pumped into the flow cell at a rate of 13 μl/min and the temperature is set to 85° C. for 60 seconds. 10 μl of “3′ Cap” is pumped into the flow cell at a rate of 13 μl/min and the temperature is set to 85° C. for 90 seconds. 10 μl of “3′ Cap” is pumped into the flow cell at a rate of 13 μl/min and the temperature is set to 85° C. for 120 seconds. 10 μl of “3′ Cap” is pumped into the flow cell at a rate of 13 μl/min and the temperature is set to 85° C. for 150 seconds. 10 μl of “3′ Cap” is pumped into the flow cell at a rate of 13 μl/min and the temperature is set to 85° C. for 180 seconds. 10 μl of “3′ Cap” is pumped into the flow cell at a rate of 13 μl/min and the temperature is set to 85° C. for 210 seconds. 10 μl of “3′ Cap” is pumped into the flow cell at a rate of 13 μl/min and the temperature is set to 85° C. for 240 seconds.

10 μl of “3′ Cap” is pumped into the flow cell at a rate of 13 μl/min and the temperature is set to 85° C. for 270 seconds. 75 μl of “Wash Buffer” is pumped into the flow cell at a rate of 60 μl/min and the temperature is set to 0° C. 200 μl of “Folding Buffer (chilled)” is pumped into the flow cell at a rate of 250 μl/min followed by 160 μl of “Folding Buffer (chilled)” at a rate of 40 μl/min and the temperature is set to 0° C. for 600 seconds.

The temperature is raised to 37° C. for 120 seconds. This is followed by a binding step.

For the binding step, 80 μl of “Binding Buffer” is pumped into the flow cell at a rate of 250 μl/min and the temperature is set to 37° C. 80 μl of “Target #1” is pumped into the flow cell at a rate of 100 μl/min and the temperature is set to 37° C. for 300 seconds. 10 μl of “Target #1” is again pumped into the flow cell at a rate of 13 μl/min and the temperature is set to 37° C. for 300 seconds. Lastly, 10 μl of “Target #1” is pumped into to the flow cell at a rate of 13 μl/min and the temperature is set to 37° C. for 2700 seconds.

This is followed by a three consecutive incorporation steps and wash steps to remove unbound target consisting of incorporation, pumping 80 μl of “Binding Buffer” into the flow cell at a rate of 13 μl/min, incorporation, pumping 80 μl of “Binding Buffer” into the flow cell at a rate of 80 μl/min, incorporation, pumping 80 μl of “Binding Buffer” into the flow cell at a rate of 200 μl/min and incorporation.

The denaturing, capping, binding, incorporation and washing steps above are repeated until sequencing and target introduction is complete. Various targets are then added and binding to and/or activity of the aptamers is evaluated.

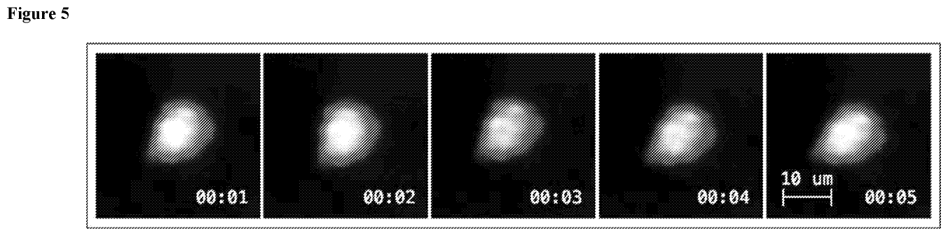

FIG. 5 shows a time lapse image of the movement of a Hana cell bound to the flow cell. The results demonstrate that the cell is actually bound by the sequences attached to the surface itself, rather than the surface itself, and is thus free to move but confined to that location.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of treating a subject for a disease associated with a rapidly evolving biological entity, the method comprising:
(a) administering to the subject a therapeutic nucleic acid that targets the rapidly evolving biological entity;
(b) determining whether the subject exhibits a therapeutic response; and
(c) if the subject fails to demonstrate a therapeutic response:
(i) obtaining a sample from the subject comprising the rapidly evolving biological entity;
(ii) performing a screening assay to identify a new therapeutic nucleic acid that targets the rapidly evolving biological entity; and
(iii) administering to the subject the new therapeutic nucleic acid.

2. The method of claim 1, wherein step (c) further comprises continuing to administer the therapeutic nucleic acid if the subject shows a therapeutic response.

3. The method of claim 2, wherein steps (b)-(c) are repeated until: (1) the disease associated with the rapidly evolving biological entity is treated; or (2) the rapidly evolving biological entity is eliminated from the subject.

4-5. (canceled)

6. The method of claim 1, further comprising, prior to step (a), performing the steps of:
(1) obtaining a sample from the subject comprising the rapidly evolving biological entity; and
(2) performing a screening assay to identify the therapeutic nucleic acid.

7. The method of claim 1, further comprising performing an analysis of the rapidly evolving biological entity prior to step (a).

8. The method of claim 7, wherein the analysis of the rapidly evolving biological entity comprises a nucleic acid sequencing analysis, a proteomic analysis, a surface marker expression analysis, a cell cycle analysis, a metabolomics analysis or analysis by direct selection of the nucleic acid without a-priori knowledge of the entity's genotype and/or phenotype.

9. A method of treating a subject for a disease associated with a rapidly evolving biological entity, the method comprising:
(a) administering to the subject a therapeutic nucleic acid that targets the rapidly evolving biological entity;
(b) after a period of time, obtaining a sample from the subject comprising the rapidly evolving biological entity;
(c) performing a screening assay to identify a new therapeutic nucleic acid that targets the rapidly evolving biological entity;
(d) administering to the subject the new therapeutic nucleic acid.

10. The method of claim 9, wherein the period of time in step (b) is equal to or shorter than the period of time required for the rapidly evolving biological entity to either acquire resistance to the therapeutic nucleic acid or to complete a replication cycle.

11. (canceled)

12. The method of claim 9, wherein steps (b)-(d) are repeated until the disease associated with the rapidly evolving biological entity is treated.

13. The method of claim 9, further comprising, prior to step (a), performing the steps of:
(1) obtaining a sample from the subject comprising the rapidly evolving biological entity;
(2) performing a screening assay to identify the therapeutic nucleic acid; and
(3) performing an analysis of the rapidly evolving biological entity prior to step (a).

14-15. (canceled)

16. The method of claim 1, wherein the rapidly evolving biological entity is a bacterium, a virus, or a cancer cell.

17-22. (canceled)

23. The method of claim 1, wherein the therapeutic nucleic acid is an interfering RNA or a nucleic acid aptamer.

24. The method of claim 23, wherein the therapeutic nucleic acid is a nucleic acid aptamer.

25-26. (canceled)

27. The method of claim 24, wherein the screening assay comprises:
(1) contacting a plurality of aptamer clusters immobilized on a surface with the rapidly evolving biological entity from the sample; and (2) identifying the immobilized aptamer clusters that specifically bind to the rapidly evolving biological entity or modulate a property of the rapidly evolving biological entity.

28. The method of claim 27, further comprising one or more of the following steps-ef:
(a) immobilizing a plurality of aptamers from an aptamer library on a surface; and
(b) amplifying the plurality of immobilized aptamers locally on the surface to form the plurality of immobilized aptamer clusters;
(c) removing the complementary strands from the immobilized aptamer clusters to provide single stranded immobilized aptamer clusters;
(d) performing a wash step after step (1) to remove unbound rapidly evolving biological entities from the surface; and
(e) sequencing the immobilized aptamers before step 1.

29-32. (canceled)

33. The method of claim 27, further comprising generating the plurality of immobilized aptamer clusters by printing aptamers from an aptamer library onto the surface.

34. The method of claim 27, wherein at least 10⁸ distinct aptamers are immobilized on the surface and each aptamer cluster comprises at least 50 identical aptamers.

35. (canceled)

36. The method of claim 27, wherein the surface is a flow cell surface.

37. (canceled)

38. The method of claim 27, wherein each of the immobilized aptamers have a sequence according to Formula II or Formula III:
P1-S1-L1-S1*-S2-L2-S2*-P2 (II), or
P1-S1-L1-S2-L2-S2*-L1-S1*-P2 (III),
wherein:
P1 is a 5′ primer site sequence;
P2 is a 3′ primer site sequence;
S1 and S2 are each independently a stem region sequence of at least one base;
S1* is a complementary sequence to S1;
S2* is a complementary sequence to S2; and
L1 and L2 are each independently a Loop region sequence of at least one base.

39. The method of claim 27, wherein the rapidly evolving biological entity is detectably labeled.

40-51. (canceled)

52. The method of claim 39, wherein the detectable label is a fluorescent dye.

53. The method of claim 52, wherein the fluorescent dye is a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, or a redox potential dye.

54. The method of claim 39, wherein the detectable label is an activation associated marker, an oxidative stress reporter, an angiogenesis marker, an apoptosis marker, an autophagy marker, a cell viability marker, or a marker for ion concentrations.

55. The method of claim 39, wherein the cell is labeled with a fluorescently-labeled antibody or antigen-binding fragment thereof, annexin V, a fluorescently-labeled fusion protein, a fluorescently-labeled sugar, or fluorescently labeled lectin.

56. (canceled)

57. The method of claim 27, wherein the property of the rapidly evolving biological entity that is modulated is cell viability, cell proliferation, gene expression, cell morphology, cellular activation, phosphorylation, calcium mobilization, degranulation or cellular migration, cellular differentiation.

58. (canceled)


SEQUENCE LISTING (4 sequences)

SEQ ID NO: 1 - DNA, 47 bp, Artificial Sequence
  source: /note=“Description of Artificial Sequence Synthetic primer”
gtcacatctcgtatgccgtcttctgcttgatccagagtgacgcagca

SEQ ID NO: 2 - DNA, 48 bp, Artificial Sequence
  source: /note=“Description of Artificial Sequence Synthetic primer”
ctctttccctacacgacgctcttccgatctactaagccaccgtgtcca

SEQ ID NO: 3 - DNA, 46 bp, Artificial Sequence
  source: /note=“Description of Artificial Sequence Synthetic primer”
gatcggaagagcacacgtctgaactccagtcacatctcgtatgccg

SEQ ID NO: 4 - DNA, 50 bp, Artificial Sequence
  source: /note=“Description of Artificial Sequence Synthetic primer”
aatgatacggcgaccaccgagatctacacacactctttccctacacgacg